(19) United States
(12) Patent Application Publication
(10) Pub. No.: US 2024/0093175 A1
(43) Pub. Date: Mar. 21, 2024

(54) PULSED ELECTRIC FIELD TREATMENT OF BIOLOGICAL CELLS

(71) Applicant: Bühler AG, Uzwil (CH)

(72) Inventor: Leandro BUCHMANN, Winterthur (CH)

(73) Assignee: Bühler AG, Uzwil (CH)

(21) Appl. No.: 18/266,274

(22) PCT Filed: Dec. 2, 2021
(86) PCT No.: PCT/EP2021/083976

§ 371 Date: Jun. 9, 2023

(30) Foreign Application Priority Data
Dec. 9, 2020 (EP) 20212860.9

(51) Int. Cl. C12N 13/00; C12N 1/16; C12N 1/20

(52) U.S. Cl. CPC C12N 13/00; C12N 1/16; C12N 1/20

(57) ABSTRACT

The present invention relates to methods of increasing metabolic activity and stimulating cell proliferation of biological cells through the use of pulsed electric field (PEF) treatment, especially through the use of nanosecond pulsed electric field (nsPEF) treatment.

### FIELD OF THE INVENTION

The present invention relates to methods of increasing metabolic activity and stimulating cell proliferation of biological cells through the use of pulsed electric field (PEF) treatment, especially through the use of nanosecond pulsed electric field (nsPEF) treatment.

### BACKGROUND OF THE INVENTION

Pulsed electric field (PEF) processing is a growing field in the area of electro-magnetic technologies for medical, environmental, and food applications (see Perspective article by Buchmann & Mathys, Frontiers in Bioengineering and Biotechnology, vol. 7, article 265, 16 Oct. 2019). PEF treatment is based on the formation of a potential difference across a conductive biological material between two electrodes, creating an electric field that depends on the applied electric voltage, the shape of the electrodes, and the gap between electrodes.

PEF processing can be divided into conventional PEF processing in the range of micro- to milliseconds and nanosecond (nsPEF) processing, in which high electric fields (10-100 kV cm⁻¹) are applied for 1-300 ns. nsPEF induces intracellular effects, distinct from the pronounced effects of conventional PEF on the cell membrane. Thereby, innovative applications and novel process windows are possible, while similar components for both treatments in batch and continuous mode are required. In both cases, the resulting electropermeabilization increases the mass transfer of molecules and ions. Depending on process parameters, a reversible or irreversible effect can be induced. Most current applications are focused on irreversible electropermeabilization, including non (minimal)-thermal pasteurization, enhanced drying rates, increased extraction yields, tissue softening as well as electrochemotherapy, and tumor ablation. Reversible electropermeabilization is typically used in molecular biology for the introduction of specific molecules, such as plasmids and antibodies, in vivo. However, the mechanisms underlying the PEF/nsPEF induced effects are still the subject of intensive research.

### Technical Problems Underlying the Present Invention

The present inventors have devised improved processes of pulsed electric field (PEF) treatment and nanosecond pulsed electric field (nsPEF) treatment of prokaryotic and eukaryotic cells.

The processes of the present invention provide an increased productivity of the treated cells due to a more homogenous cellular response; allow for both an intracellular and an extracellular manipulation of cells; result in an increased biomass yield and an increased production of specific compounds due to a more targeted and more ubiquitous application of the electric field; allows specific manipulation of cells in a wide range of sizes; and can be easily adapted to an industrial scale.

This summary does not necessarily describe all advantages achieved and all problems solved by the present invention.

### SUMMARY OF THE INVENTION

In a first aspect the present invention relates to a method of increasing metabolic activity of biological cells, said method comprising the following steps:
(a) providing biological cells and suspending the cells in an electrically conductive liquid, thereby forming a suspension,
(b) positioning the suspension between two electrodes, and
(c) applying from 1 to 100 pulses, preferably from 2 to 30 pulses, of electricity between the electrodes, thereby increasing metabolic activity of the biological cells;
   wherein a voltage increase between the two electrodes from 10% to 90% of a target voltage of the pulses of electricity takes place within a time period of 0.1 to 100 ns, preferably within a time period of 1 ns to 25 ns; wherein the pulses of electricity have a pulse duration of between 5 to 5000 ns, preferably of between 50 ns to 300 ns; and wherein the pulses of electricity, when reaching the target voltage, have an electric field strength of 0.5 kV/cm to 50 kV/cm, preferably of 1.0 kV/cm to 30 kV/cm.

In a second aspect the present invention relates to a method of stimulating cell proliferation and/or increasing metabolic activity of biological cells, said method comprising the following steps:
(a) providing biological cells and suspending the cells in an electrically conductive liquid, thereby forming a suspension,
(b) positioning the suspension between two electrodes, and
(c) applying from 2 to 30 pulses, preferably from 2 to 12 pulses, of electricity between the electrodes, thereby stimulating cell proliferation;
   wherein a voltage increase between the two electrodes from 10% to 90% of a target voltage of the pulses of electricity takes place within a time period of 1 ns to 25 ns, preferably within a time period of 3 ns to 15 ns; wherein the pulses of electricity have a pulse duration of between 50 ns to 300 ns, preferably of about 100 ns; and wherein the pulses of electricity, when reaching the target voltage, have an electric field strength of 1.0 kV/cm to 30 kV/cm, preferably of 5.0 kV/cm to 20 kV/cm.

In a third aspect the present invention relates to a method of stimulating cell proliferation and/or increasing metabolic activity of biological cells, said method comprising the following steps:
(a) providing biological cells and suspending the cells in an electrically conductive liquid, thereby forming a suspension,
(b) positioning the suspension between two electrodes, and
(c) applying from 1 to 100 pulses, preferably from 2 to 30 pulses, of electricity between the electrodes, thereby stimulating cell proliferation;
   wherein a voltage increase between the two electrodes from 10% to 90% of a target voltage of the pulses of electricity takes place within a time period of 0.1 to 100 ns, preferably within a time period of 1 ns to 25 ns; wherein the pulses of electricity have a pulse duration of between 5 to 5000 ns, preferably of between 50 ns to 300 ns; and wherein the pulses of electricity, when reaching the target voltage, have an electric field strength of 0.5 kV/cm to 50 kV/cm, preferably of 1.0 kV/cm to 30 kV/cm; wherein the biological cells are selected from the group consisting of bacterial cells, yeast cells, fungal cells, microalgae cells, plant cells, animal cells, and human cells.

In a fourth aspect the present invention relates to a method of stimulating cell proliferation and/or increasing metabolic activity of biological cells, said method comprising the following steps:
(a) providing biological cells and suspending the cells in an electrically conductive liquid, thereby forming a suspension,
(b) positioning the suspension between two electrodes, and
(c) applying from 1 to 100 pulses, preferably from 2 to 30 pulses, of electricity between the electrodes, thereby stimulating cell proliferation;
   wherein a voltage increase between the two electrodes from 10% to 90% of a target voltage of the pulses of electricity takes place within a time period of 0.1 to 100 ns, preferably within a time period of 1 ns to 25 ns; wherein the pulses of electricity have a pulse duration of between 5 to 5000 ns, preferably of between 50 ns to 300 ns; and wherein the pulses of electricity, when reaching the target voltage, have an electric field strength of 0.5 kV/cm to 50 kV/cm, preferably of 1.0 kV/cm to 30 kV/cm;
wherein step (c) is carried out in a recirculation process.

In a fifth aspect the present invention relates to a method of stimulating cell proliferation and/or increasing metabolic activity of biological cells, said method comprising the following steps:
(a) providing biological cells and suspending the cells in an electrically conductive liquid, thereby forming a suspension,
(b) positioning the suspension between two electrodes, and
(c) applying from 1 to 100 pulses, preferably from 2 to 30 pulses, of electricity between the electrodes, thereby stimulating cell proliferation;
   wherein a voltage increase between the two electrodes from 10% to 90% of a target voltage of the pulses of electricity takes place within a time period of 0.1 to 100 ns, preferably within a time period of 1 ns to 25 ns; wherein the pulses of electricity have a pulse duration of between 5 to 5000 ns, preferably of between 50 ns to 300 ns; and wherein the pulses of electricity, when reaching the target voltage, have an electric field strength of 0.5 kV/cm to 50 kV/cm, preferably of 1.0 kV/cm to 30 kV/cm;
wherein step (c) is carried out in a batch process.

### DETAILED DESCRIPTION OF THE INVENTION

### Definitions

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

As used herein, the term “about” refers to numerical values ranging from 5% below the indicated numerical value to 5% above the indicated numerical value. For example, a pulse duration of “about 100 ns” encompasses a pulse duration ranging from 95 ns to about 105 ns.

As used herein, the term “cells” refers both to single cells and to aggregates of two or more cells (e.g. up to 10 cells, up to 100 cells, up to 1000 cells, up to 10,000 cells, or even to large aggregates of cells comprising more than 10,000 cells). In the context of the present invention, the only upper limitation for the size of the cell aggregates is that the aggregate should be sufficiently small to prepare a suspension of the cell aggregate in the electrically conductive liquid described herein.

Exemplary embodiments of single cells include, without limitation, cells of unicellular organisms (e.g. bacterial cells, yeast cells, or microalgae) or isolated cells from multicellular organisms (e.g. isolated cells from plants, fungi, animals or humans; in particular isolated somatic cells from animals or humans or induced pluripotent stem cells (iPSCs) from animals or humans). Aggregates of two or more cells are derived from multicellular organisms. Exemplary embodiments of such aggregates include, without limitation, multicellular tissue samples, such as meristems in plants or epithelial or connective tissue from animals or humans.

### Embodiments of the Invention

The present invention will now be further described. In the following passages different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

In a first aspect the present invention is directed to a method of increasing metabolic activity of biological cells, said method comprising the following steps:
(a) providing biological cells and suspending the cells in an electrically conductive liquid, thereby forming a suspension,
(b) positioning the suspension between two electrodes, and
(c) applying from 1 to 100 pulses of electricity between the electrodes, thereby increasing metabolic activity of the biological cells;
   wherein a voltage increase between the two electrodes from 10% to 90% of a target voltage of the pulses of electricity takes place within a time period of 0.1 to 100 ns, wherein the pulses of electricity have a pulse duration of between 5 to 5000 ns, and wherein the pulses of electricity, when reaching the target voltage, have an electric field strength of 0.5 kV/cm to 50 kV/cm.

The present invention demonstrates that the described processes of PEF treatment are not only suitable to stimulate proliferation of biological cells but are also suitable to increase metabolic activity of biological cells and achieve higher yields of specific endogenous or exogenous compounds.

As will be clear to a person of skill in the art, “achieving higher yields” includes the aspects of
obtaining more biomass and/or more desired product in the same time; or
obtaining the same amount of biomass and/or the same amount of product in a shorter time;
when compared to processes without PEF treatment.

In a second aspect the present invention is directed to a method of stimulating cell proliferation and/or increasing metabolic activity of biological cells, said method comprising the following steps:
(a) providing biological cells and suspending the cells in an electrically conductive liquid, thereby forming a suspension,
(b) positioning the suspension between two electrodes, and
(c) applying from 2 to 30 pulses of electricity between the electrodes, thereby stimulating cell proliferation;
   wherein a voltage increase between the two electrodes from 10% to 90% of a target voltage of the pulses of electricity takes place within a time period of 1 ns to 25 ns, wherein the pulses of electricity have a pulse duration of between 50 ns to 300 ns, and wherein the pulses of electricity, when reaching the target voltage, have an electric field strength of 1.0 kV/cm to 30 kV/cm.

Without wishing to be bound by a particularly theory, it is the inventors' opinion that the physical parameters used in the second aspect will lead to an increased productivity due to a more homogenous cellular response; and/or will allow both an intracellular and an extracellular manipulation of cells; and/or will result in an increased biomass yield and/or an increased production of specific compounds due to a more targeted and more ubiquitous application of the electric field; and/or will allow specific manipulation of cells in the range from 1 μm to 70 mm in size (preferably from 1 μm to 10 mm); and/or will allow the adaptation of the process to an industrial scale.

In a third aspect the present invention is directed to a method of stimulating cell proliferation and/or increasing metabolic activity of biological cells, said method comprising the following steps:
(a) providing biological cells and suspending the cells in an electrically conductive liquid, thereby forming a suspension,
(b) positioning the suspension between two electrodes, and
(c) applying from 1 to 100 pulses of electricity between the electrodes, thereby stimulating cell proliferation;
   wherein a voltage increase between the two electrodes from 10% to 90% of a target voltage of the pulses of electricity takes place within a time period of 0.1 to 100 ns, wherein the pulses of electricity have a pulse duration of between 5 to 5000 ns, and wherein the pulses of electricity, when reaching the target voltage, have an electric field strength of 0.5 kV/cm to 50 kV/cm;
wherein the biological cells are selected from the group consisting of bacterial cells, yeast cells, fungal cells, microalgae cells, plant cells, animal cells, and human cells.

In a fourth aspect the present invention is directed to a method of stimulating cell proliferation and/or increasing metabolic activity of biological cells, said method comprising the following steps:
(a) providing biological cells and suspending the cells in an electrically conductive liquid, thereby forming a suspension,
(b) positioning the suspension between two electrodes, and
(c) applying from 1 to 100 pulses of electricity between the electrodes, thereby stimulating cell proliferation;
   wherein a voltage increase between the two electrodes from 10% to 90% of a target voltage of the pulses of electricity takes place within a time period of 0.1 to 100 ns, wherein the pulses of electricity have a pulse duration of between 5 to 5000 ns, and wherein the pulses of electricity, when reaching the target voltage, have an electric field strength of 0.5 kV/cm to 50 kV/cm;
wherein step (c) is carried out in a recirculation process.

Without wishing to be bound by a particularly theory, it is the inventors' opinion that a recirculation process facilitates the industrial implementation of the process of pulsed electric filed treatment.

In a fifth aspect the present invention is directed to a method of stimulating cell proliferation and/or increasing metabolic activity of biological cells, said method comprising the following steps:
(a) providing biological cells and suspending the cells in an electrically conductive liquid, thereby forming a suspension,
(b) positioning the suspension between two electrodes, and
(c) applying from 1 to 100 pulses of electricity between the electrodes, thereby stimulating cell proliferation;
   wherein a voltage increase between the two electrodes from 10% to 90% of a target voltage of the pulses of electricity takes place within a time period of 0.1 to 100 ns, wherein the pulses of electricity have a pulse duration of between 5 to 5000 ns, and wherein the pulses of electricity, when reaching the target voltage, have an electric field strength of 0.5 kV/cm to 50 kV/cm;
wherein step (c) is carried out in a batch process.

Without wishing to be bound by a particularly theory, it is the inventors' opinion that a batch process will reduce the overall mechanical and/or physical stress occurring during the electric field treatment of the cells.

In a preferred embodiment of the first, third, fourth, or fifth aspect of the invention, from 2 to 30 pulses of electricity are applied in step (c).

In a preferred embodiment of any aspect of the invention, from 2 to 25 pulses, preferably from 2 to 20 pulses, more preferably from 2 to 15 pulses, and even more preferably from 2 to 12 pulses, for example 2, 3, 4, 5, 6, 7, 8, 9, 10, 11, or 12 pulses, are applied in step (c).

In a preferred embodiment of the first, third, fourth, or fifth aspect of the invention, the voltage increase between the two electrodes from 10% to 90% of a target voltage of the pulses of electricity takes place within a time period of 1 ns to 25 ns,

In a preferred embodiment of any aspect of the invention, the voltage increase between the two electrodes from 10% to 90% of a target voltage of the pulses of electricity takes place within a time period of 2 ns to 10 ns, more preferably within a time period of 3 ns to 15 ns.

In a preferred embodiment of the first, third, fourth, or fifth aspect of the invention, the pulses of electricity have a pulse duration of between 50 ns and 300 ns.

In a preferred embodiment of any aspect of the invention, the pulses of electricity have a pulse duration of between 60 ns and 250 ns, preferably of between 70 ns and 200 ns, more preferably of between 80 ns and 150 ns, more preferably of between 90 ns and 125 ns, and most preferably of about 100 ns.

In a preferred embodiment of the first, third, fourth, or fifth aspect of the invention, the pulses of electricity, when reaching the target voltage, have an electric field strength of 1.0 kV/cm to 30 kV/cm.

In a preferred embodiment of any aspect of the invention, the pulses of electricity, when reaching the target voltage, have an electric field strength of 2 kV/cm to 28 kV/cm, preferably 3 kV/cm to 25 kV/cm, more preferably 4 kV to 23 kV/cm, and most preferably of 5 kV/cm to 20 kV/cm.

In a preferred embodiment of the first, third, fourth, or fifth aspect of the invention, the potential difference between the electrodes is between 2 to 300 kV, preferably between 2 and 250 kV, more preferably between 2 and 200 kV, more preferably between 2 and 150 kV, more preferably, between 2 and 100 kV, more preferably between 2 and 50 kV, even more preferably between 2 and 40 kV, even more preferably between 3 and 30 kV, even more preferably between 4 and 20 kV, and most preferably between 5 and 10 kV.

In a preferred embodiment of the first, second, fourth, or fifth aspect of the invention, the biological cells are selected from the group consisting of bacterial cells, yeast cells, fungal cells, microalgae cells, plant cells, animal cells, and human cells.

In a preferred embodiment of any aspect of the invention,
the bacterial cells are selected from the group consisting of Escherichia coli and Corynebacterium glutamicum;
the yeast cells are selected from the group consisting of Pichia pastoris and Saccharomyces cerevisiae;
the microalgae cells are selected from the group consisting of Galdieria sulphuraria and Aurantiochytrium limacinum;
the plant cells are selected from the group consisting of Hordeum vulgare and Oryza sativa;
the animal cells are selected from the group consisting of Spodoptera frugiperda, Trichoplusia ni, Bos taurus, Sus scrofa (domesticus), Gallus galls (domesticus), Ovis gmelini aries, and Capra aegragus hircus.

In a preferred embodiment of the first, second, or third aspect of the invention, step (c) is carried out in a recirculation process.

In a preferred embodiment of the first, second, or third aspect of the invention, step (c) is carried out in a batch process.

The following FIGURES and examples are merely illustrative of the present invention and should not be construed to limit the scope of the invention as indicated by the appended claims in any way.

### BRIEF DESCRIPTION OF THE FIGURES

FIG. 1.: Metabolic activity of different E. coli samples determined by FDA (fluorescein diacetate) cleavage. The E. coli cell samples received different numbers of electric pulses of a nanosecond pulsed electric field (nsPEF) treatment in a recirculation process. All E. coli cell samples exhibited an increase in metabolic activity due to the nsPEF treatment.

### EXAMPLES

### Example 1

A twin bioreactor system was used for parallel cultivations. The parallel cultivations were inoculated with the same amount of pre-culture from the same feed train of the selected bacterial cell Escherichia coli. The reactor cultivations as well as the pre-culture/inoculum were performed in the modified, defined MD-FB medium:
14.3 g L⁻¹ (pre-culture) and 28.7 g L⁻¹ glycerol 85%,
K₂HPO₄ 2.70 g L⁻¹,
KH₂PO₄ 13.2 g L⁻¹,
NaCl 2.04 g L⁻¹,
(NH₄)SO₄ 4.1 g L⁻¹,
Antifoam 205 0.05 g L⁻¹,
ddH₂O 920.8 g L⁻¹,
0.8 mL MgSO₄*7 H₂O stock (300 g L⁻¹),
10.0 mL Fe(III) citrate stock (10.0 g L⁻¹),
10 mL Na-EDTA*2 H₂O stock (0.84 g L⁻¹),
2.8 mL Thiamine HCl stock (45.0 g L⁻¹),
2.9 mL TE stock (COCl₂*6 H₂O 0.16 g L⁻¹,
MnCl₂*4 H₂O 1.42 g L⁻¹,
H₃BO₃ 0.01 g L⁻¹,
Na₂MoO₄*2 H₂O 0.02 g L⁻¹,
CaCl₂)*2 H₂O 1.44 g L⁻¹,
AlCl₃*6 H₂O 0.04 g L⁻¹,
ZnSO₄*7 H₂O 0.87 g L¹,
CuSO₄*5 H₂O 1.55 g L⁻¹,
NiCl₂*6 H₂O 0.01 g L⁻¹

Cultivation conditions are summarized in Table 1:

TABLE 1

| Parameter | Value | Unit |
|---|---|---|
| Reactor working volume | 10 | [L] |
| pH | 7 | [—] |
| Aeration (sterile air) | 3 | [vvm] |
| Temperature | 30.0 | [° C.] |
| Pressure | 0.5 | [bar] |
| Stirrer | 1200 | [rpm] |

Upon treatment the metabolic activity was expressed as the rate and total amount of individual cellular FDA (fluorescein diacetate) cleavage. Standard staining protocols were used in the experiments and analysed in a flow cytometer (Ehgartner D, Herwig C, Neutsch L. At-line determination of spore inoculum quality in Penicillium chrysogenum bioprocesses. Appl Microbiol Biotechnol. 2016; 100(12):5363-73; Söderström BE. Vital staining of fungi in pure cultures and in soil with fluorescein diacetate. Soil Biol Biochem. 1977; 9:59-63; and Pekarsky, A., Veiter, L., Rajamanickam, V. et al. Production of a recombinant peroxidase in different glyco-engineered Pichia pastoris strains: a morphological and physiological comparison. Microb Cell Fact 17, 183 (2018)).

Table 2 summarizes the nsPEF conditions for the control and five different samples.

TABLE 2

| Sample No. | 010 | 012 | 016 | 018 | 019 | 0112 |
|---|---|---|---|---|---|---|
| Potential difference [kV] | Control | 10 | 10 | 10 | 10 | 10 |
| Pulse length [ns] | | 100 | 100 | 100 | 100 | 100 |
| Pulses of electricity per | | 4 | 6 | 8 | 10 | 12 |
| treatment passage [—] | | | | | | |
| Electrode distance [cm] | 0.5 | 0.5 | 0.5 | 0.5 | 0.5 | 0.5 |

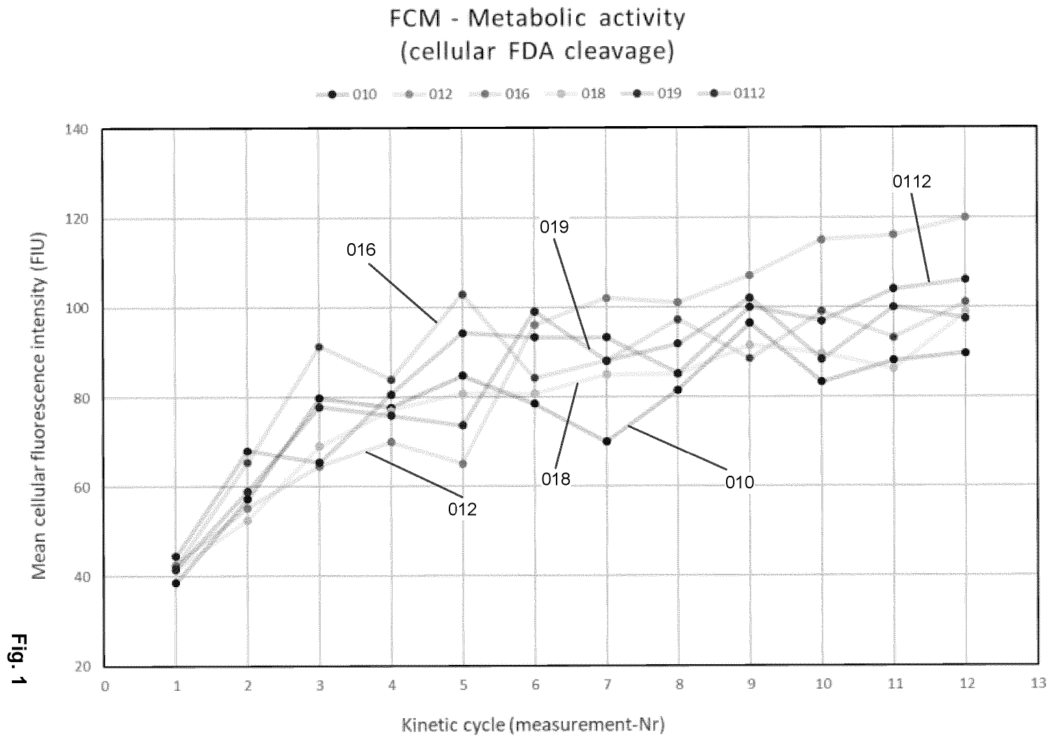

An increase in metabolic activity was detected for all treated samples (see FIG. 1). Surprisingly, the greatest increase of metabolic activity was observed for sample no. 12, which received the lowest number of pulses (i.e. 4 pulses). Sample No. 012 exhibited an increase of metabolic activity as compared to control sample 010 from 90 FIU to 120 FIU, which corresponds to an increase of about 30%

### Example 2

A twin bioreactor system was used for parallel cultivations. The parallel cultivations were inoculated with the same amount of pre-culture from the same feed train of selected yeast cell Pichia pastoris. The reactor cultivations were performed in the modified, defined medium (Hellwig et al., 2001):
35.29 g L⁻¹ Glycerol 85%,
K₂SO₄ 2.86 g L⁻¹,
KOH 0.64 g L⁻¹,
MgSO₄*7 H₂O 2.32 g L⁻¹,
CaSO₄*2 H₂O 0.17 g L⁻¹,
Na-EDTA*2 H₂O 0.6 g L⁻¹,
NaCl 0.22 g L⁻¹,
Antifoam 205 0.1 g L⁻¹,
H₃PO₄ (85% 7.19 g L⁻¹,
Water 860 g L⁻¹,
PTM1 stock 4.35 mL L⁻¹ (CuSO₄ 6.0 g L⁻¹, NaI 0.08 g L⁻¹, MnSO₄*H₂O 3.0 g L⁻¹, Na₂MoO₄*2 H₂O 0.2 g L⁻¹, CoCl₂*6 H₂O 0.5 g L⁻¹, ZnCl₂ 20.0 g L⁻¹, FeSO₄*7 H₂O 65.0 g L⁻¹, H₂SO₄ (98%) 50 mL l⁻¹, H₃BO₃ 0.02 g L⁻¹),
Biotin stock 500×4.35 mL L⁻¹ (0.2 g L⁻¹)

Cultivation conditions are summarized in Table 3:

TABLE 3

| Parameter | Value | Unit |
|---|---|---|
| Reactor working volume | 10 | [L] |
| pH | 7 | [—] |
| Aeration (sterile air) | 3 | [vvm] |
| Temperature | 30.0 | [° C.] |
| Pressure | 0.5 | [bar] |
| Stirrer | 800 | [rpm] |

Upon treatment the metabolic activity was increased on an individual cell basis, based on the analysed parameters.

Table 4 summarizes the nsPEF conditions for the control and four different samples.

TABLE 4

| | Sample No. | | | | |
|---|---|---|---|---|---|
| | 0 | 1 | 2 | 3 | 4 |
| Potential difference [kV] | Control | 5 | 5 | 5 | 5 |
| Pulse length [ns] | | 100 | 100 | 100 | 100 |
| Pulses of electricity per | | 2 | 3 | 4 | 6 |
| treatment passage [—] | | | | | |
| Electrode distance [cm] | 0.5 | 0.5 | 0.5 | 0.5 | 0.5 |

1. An in vitro method of increasing metabolic activity of biological cells, said method comprising the following steps:
(a) suspending biological cells in an electrically conductive liquid, thereby forming a suspension,
(b) positioning the suspension between two electrodes, and
(c) applying from 1 to 100 pulses, preferably from 2 to 30 pulses, of electricity between the electrodes, thereby increasing metabolic activity of the biological cells;
characterized in that a voltage increase between the two electrodes from 10% to 90% of a target voltage of the pulses of electricity takes place within a time period of 0.1 to 100 ns, preferably within a time period of 1 ns to 25 ns; wherein the pulses of electricity have a pulse duration of between 5 to 5000 ns, preferably of between 50 ns to 300 ns; and wherein the pulses of electricity, when reaching the target voltage, have an electric field strength of 0.5 kV/cm to 50 kV/cm, preferably of 1.0 kV/cm to 30 kV/cm.

2. The method of claim 1, wherein the biological cells are selected from the group consisting of bacterial cells, yeast cells, fungal cells, microalgae cells, plant cells, animal cells, and human cells.

3. An in vitro method of stimulating cell proliferation and/or increasing metabolic activity of biological cells, said method comprising the following steps:
(a) suspending biological cells in an electrically conductive liquid, thereby forming a suspension,
(b) positioning the suspension between two electrodes, and
(c) applying from 1 to 100 pulses, preferably from 2 to 30 pulses, of electricity between the electrodes, thereby stimulating cell proliferation;
characterized in that a voltage increase between the two electrodes from 10% to 90% of a target voltage of the pulses of electricity takes place within a time period of 0.1 to 100 ns, preferably within a time period of 1 ns to 25 ns; wherein the pulses of electricity have a pulse duration of between 5 to 5000 ns, preferably of between 50 ns to 300 ns; and wherein the pulses of electricity, when reaching the target voltage, have an electric field strength of 0.5 kV/cm to 50 kV/cm, preferably of 1.0 kV/cm to 30 kV/cm;
wherein the biological cells are selected from the group consisting of bacterial cells, yeast cells, fungal cells, microalgae cells, plant cells, and animal cells, wherein the animal cells are selected from the group consisting of Spodoptera frugiperda and Trichoplusia ni.

4. The method of claim 2, wherein
the bacterial cells are selected from the group consisting of Escherichia coli and Corynebacterium glutamicum;
the yeast cells are selected from the group consisting of Pichia pastoris and Saccharomyces cerevisiae;
the microalgae cells are selected from the group consisting of Galdieria sulphuraria and Aurantiochytrium limacinum;
the plant cells are selected from the group consisting of Hordeum vulgare and Oryza sativa; and
the animal cells are selected from the group consisting of Spodoptera frugiperda and Trichoplusia ni.

5. The method of claim 1, wherein step (c) is carried out in a recirculation process.

6. The method of claim 1, wherein step (c) is carried out in a batch process.

7. An in vitro method of stimulating cell proliferation and/or increasing metabolic activity of biological cells, said method comprising the following steps:
(a) suspending biological cells in an electrically conductive liquid, thereby forming a suspension,
(b) positioning the suspension between two electrodes, and
(c) applying from 1 to 100 pulses, preferably from 2 to 30 pulses, of electricity between the electrodes, thereby stimulating cell proliferation;
characterized in that a voltage increase between the two electrodes from 10% to 90% of a target voltage of the pulses of electricity takes place within a time period of 0.1 to 100 ns, preferably within a time period of 1 ns to 25 ns; wherein the pulses of electricity have a pulse duration of between 5 to 5000 ns, preferably of between 50 ns to 300 ns; and wherein the pulses of electricity, when reaching the target voltage, have an electric field strength of 0.5 kV/cm to 50 kV/cm, preferably of 1.0 kV/cm to 30 kV/cm;
wherein step (c) is carried out in a recirculation process.

8. The method of claim 7, wherein the biological cells are selected from the group consisting of bacterial cells, yeast cells, fungal cells, microalgae cells, plant cells, animal cells, and human cells.

9. The method of claim 8, wherein
the bacterial cells are selected from the group consisting of Escherichia coli and Corynebacterium glutamicum;
the yeast cells are selected from the group consisting of Pichia pastoris and Saccharomyces cerevisiae;
the microalgae cells are selected from the group consisting of Galdieria sulphuraria and Aurantiochytrium limacinum;
the plant cells are selected from the group consisting of Hordeum vulgare and Oryza sativa; and
the animal cells are selected from the group consisting of Spodoptera frugiperda and Trichoplusia ni.
